(19) United States
(12) Patent Application Publication
(10) Pub. No.: US 2002/0019048 A1
(43) Pub. Date: Feb. 14, 2002

(54) Methods for restoring or enhancing T-cell immune surveillance following naturally or artificially induced immunosuppression

(76) Inventor: Ronald Berenson, Mercer Island, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/864,930

(22) Filed: May 24, 2001

Related U.S. Application Data
(60) Provisional application No. 60/207,120, filed on May 25, 2000.

(51) Int. Cl. C12N 5/08; A61K 45/00

(52) U.S. Cl. 435/372; 424/93.7; 424/144.1

(57) ABSTRACT

The present invention relates generally to methods for restoring or enhancing T-cell immune surveillance, and more particularly, to a method treating an immuno-compromised, immuno-deficient, or immuno-suppressed animal by infusion of peripheral blood lymphocytes, T-cells, or activated T-cells.

### CROSS-REFERENCE TO RELATED APPLICATION

The present application claims priority to U.S. Provisional Application No. 60/207,120, filed May 25, 2000.

### TECHNICAL FIELD

The present invention relates generally to methods for restoring or enhancing T-cell immune surveillance, and more particularly, to methods for treating an immuno-compromised, immuno-deficient, or immuno-suppressed animal by infusion of peripheral blood lymphocytes, T-cells, or activated T-cells.

### BACKGROUND OF THE INVENTION

Methods of restoring or enhancing immune responses in immuno-compromised or deficient animals have many applications. For example, a variety of diseases are characterized by the development of progressive immunosuppression in a patient. The presence of an impaired immune response in patients with malignancies has been particularly well documented. Cancer patients and tumor-bearing mice have been shown to have a variety of altered immune functions such as a decrease in delayed type hypersensitivity, a decrease in lytic function and proliferative response of lymphocytes. S. Broder et al., N. Engl. J. Ned., 299: 1281 (1978); E. M. Hersh et al., N. Engl. J. Med., 273: 1006 (1965); North and Burnauker, (1984). Many other diseases or interventions are also characterized by the development of an impaired immune response. For example, progressive immunosuppression has been observed in patients with acquired immunodeficiency syndrome (AIDS), sepsis, leprosy, cytomegalovirus infections, malaria, and the like, as well as with chemotherapy and radiotherapy. The mechanisms responsible for the down-regulation of the immune response, however, remain to be fully elucidated.

The immune response is a complex phenomenon. T lymphocytes (T-cells) are critical in the development of all cell-mediated immune reactions. Helper T-cells control and modulate the development of immune responses. Cytotoxic T-cells (killer T-cells) are effector cells which play an important role in immune reactions against intracellular parasites and viruses by means of lysing infected targeT-cells. Cytotoxic T-cells have also been implicated in protecting the body from developing cancers through an immune surveillance mechanism. T suppressor cells block the induction and/or activity of T helper cells. T-cells do not generally recognize free antigen, but recognize it on the surface of other cells. These other cells may be specialized antigen-presenting cells capable of stimulating T cell division or may be virally-infected cells within the body that become a target for cytotoxic T-cells.

Cytotoxic or suppressor T-cells usually recognize antigen in association with class I Major Histocompatibility Complex (MHC) products which are expressed on all nucleated cells. Helper T-cells, and most T-cells which proliferate in response to antigen in vitro, recognize antigen in association with class II MHC products. Class II products are expressed mostly on antigen-presenting cells and on some lymphocytes. T-cells can be also divided into two major subpopulations on the basis of their cell membrane glycoproteins as defined with monoclonal antibodies. The CD4+ subset which expresses a 62 kD glycoprotein usually recognizes antigen in the context of class II antigens, whereas the CD8+ subset expresses a 76 Kd glycoprotein and is restricted to recognizing antigen in the context of Class I MHC.

Augmentation of the immune response in immune compromised animal via infusions of lymphokines, adoptive immunotherapy has met with variable and limited success. Methods are needed to improve this type of treatment. For example, lymphocyte, blood and other cell infusions are provided to immunodeficient patients in certain settings. However, provision of activated, functional cells with sufficient longevity could provide significant increased benefit and efficacy to the patient.

Accordingly, a need exists for a method by which the immunosuppressed state of T lymphocytes during disease progression can be circumvented or reversed so that the T cell immune response in the patient can develop or be augmented.

Lymphocyte, blood and other cell infusions are provided to immunodeficient patients in certain settings. However, provision of activated, functional cells with sufficient longevity could provide significant increased benefit and efficacy to the patient.

The present invention provides such methods, as well as providing other related advantages.

### SUMMARY OF THE INVENTION

The infusion of lymphocytes, T-cells, activated T-cells, activated and gene modified T-cells, activated and otherwise modulated T-cells into an immuno-deficient patient can provide immediate restoration of some level of immune function to help prevent pathogenic insults and reduce vulnerability during a temporary or even permanent immunosuppressed state. Immuno-deficiency could be natural or could be induced by a medical condition or induced by a physician during treatment for a medical condition, such as chemotherapy during treatment of cancer.

Such cells may be obtained from a patient before they are treated with regimens that result in immuno-deficiency (e.g., radiotherapy or chemotherapy) or imimunosuppression. Under conditions of pre-existing immuno-deficiency, and where a patient's own T-cells are too few to provide such a source of cells, or whose T-cells are already compromised with respect to immune function, allogeneic donor T-cells could provide immune function following adoptive transfer to the patient.

In one aspect of the present invention, such cells may be isolated and reinfused without further processing or they may be activated ex vivo using co-stimulatory pathways such as CD333 CD28-based stimulation (such as the XCELLERATE™ process by Xcyte Therapies, Seattle, Wash.) or derivatives/improvements thereof. Such activated T-cells may also have further modifications or additives included to modulate T cell function and/or increase survival and longevity upon re-infusion. Such further modifications could include gene therapy, gene modification, delivery of cytokines or other modulators of immune function, vaccines or monoclonal antibodies. Such delivery of other molecules could be intracellular (i.e., within the infused T cell (see, e.g., PCT/US96/06200, incorporated by reference)), as a component of the infusion medium, or could be administered prior to or subsequent to the infusion of T-cells.

### DETAILED DESCRIPTION OF THE INVENTION

Prior to setting forth the invention, it may be helpful to an understanding thereof to set forth definitions of certain terms that will be used hereinafter.

A “bead” refers to a microparticle capable of having immobilized thereon a first and second agent. Further, such microparticles useful within the context of the present invention include, for example, commercially available beads such as DYNABEADS™, Dynal Corporation.

The term “activation”, as used herein, refers to the state of a T-cell in which a T-cell response has been initiated by a primary signal, such as binding through the TCR/CD3 complex, but not necessarily due to interaction with a protein antigen. As such, this activation in the absence of a co-stimulatory signal leads to clonal anergy.

A “co-stimulatory signal”, as used herein, refers to a signal, which in combination with the primary signal, leads to T-cell proliferation.

As used herein, “antibody” includes both polyclonal, monoclonal antibodies, humanized and chimeric, and may be an intact molecule, a fragment thereof (such as scFv, Fv, Fd, Fab, Fab′ and F(ab)′₂ fragments), or multimers or aggregates of intact molecules and/or fragments; and may occur in nature or be produced, e.g., by immunization, synthesis or genetic engineering.

A “humanized antibody” refers to an antibody derived from a non-human antibody (typically murine), or derived from a chimeric antibody, that retains or substantially retains the antigen-binding properties of the parent antibody but which is less immunogenic in humans. This may be achieved by various methods, including by way of example: (a) grafting only the non-human CDRs onto human framework and constant regions (humanization), or (b) transplanting the entire non-human variable domains, but “cloaking” them with a human-like surface by replacement of surface residues (“veneering”). Such methods are disclosed, for example, in Jones et al., Nature 321:522-525, 1986; Morrison et al., Proc. Natl. Acad. Sci. 81:6851-6855, 1984; Morrison and Oi, Adv. Immunol. 44:65-92, 1988; Verhoeyer et al., Science 239:1534-1536, 1988; Padlan, Molec. Immun. 28:489-498, 1991; Padlan, Molec. Immun. 31(3):169-217, 1994. In the present invention, humanized antibodies include “humanized” and “veneered” antibodies. A preferred method of humanization comprises alignment of the non-human heavy and light chain sequences to human heavy and light chain sequences, selection and replacement of the non-human framework with a human framework based on such alignment, molecular modeling to predict conformation of the humanized sequence and comparison to the conformation of the parent antibody, followed by repeated back mutation of residues in the CDR region which disturb the structure of the CDRs until the predicted conformation of the humanized sequence model closely approximates the conformation of the non-human CDRs of the parent non-human antibody.

A “chimeric antibody”, as used herein, refers to an antibody containing sequences derived from two different antibodies (e.g., U.S. Pat. Nos. 4,816,567 and 5,776,456), which typically are of different species. Most typically chimeric antibodies comprise human and murine antibody fragments, generally human constant and murine variable regions.

A “ligand/anti-ligand pair”, as used herein, refers to a complementary/anticomplementary set of molecules that demonstrate specific binding, generally of relatively high affinity. Exemplary ligand/anti-ligand pairs enzyme/inhibitor, hapten/antibody, lectin/carbohydrate, ligand/receptor, and biotin/avidin or streptavidin. Within the context of the present invention specification CD3 and CD28 are anti-ligands, while agents (e.g., antibodies and antibody fragments) reactive therewith are considered ligands.

“Separation” as used herein, includes any means of substantially purifying one component from another (e.g., by filtration or magnetic attraction).

“Quiescent” as used herein, refers to a cell state wherein the cell is not actively proliferating.

A. Treatment of Immuno-Compromised States

As noted above, many animals, such as humans may suffer from immunodeficiency as a result of any of the following conditions: 1) primary immunodeficiency—examples include congenital immunodeficiencies such as adenosine deaminase deficiency, Wiskott Aldrich syndrome, or chronic mucocutaneous candidiasis, 2) secondary immunodeficiencies including those caused by treatment with anti-lymphocyte antibodies (e.g., CAMPATH), chemotherapy, radiation, or immunosuppressant agents, 3) patients with infectious diseases such as HIV, 4) patients with chronic diseases that are characterized by immune dysfunction including cancer, chronic renal failure and diabetes, 5) elderly patients who often suffer from immune dysfunction. The present invention involves treating these patients with T-cells to correct their immnunodeficient state. Treatment can be made with peripheral blood mononuclear cells, lymphocytes, T-cells, activated T-cells or T-cells activated with antibodies and/or cytokines, T-cells activated with ligands such as antibodies that target CD3 and CD28 receptors.

Cells are administered to patients at various time points to increase the level of functional lymphocytes. Administration of cells should improve the immune status of the patient and reduce their risk of infections. Treatment may comprise the cells administered alone or with cytokines such as IL-2 to enhance the activity of the administered cells. Additionally, cells may be administered prior to, during, or after vaccination to improve immune responses to a vaccine.

Previously, physicians and scientists have used granulocyte transfusions and drugs such as Neuupogen that increase neutrophil counts to reduce the risk of infections in patients with low neutrophil counts. However, there has not been a method to reduce the risk of infections in patients that suffer from lymphocyte dysfunction and/or decreased lymphocyte counts. The present invention solves this problem. Moreover, sources of cells may include the patient's own cells, allogeneic cells, or potentially xenogeneic cells. Additionally, cells may be obtained from blood but it is also possible to obtain cells from lymph node, spleen, or other organs of the immune system.

Restoration and/or enhancement of immunity in immuno-deficient;/immuno-compromised/immuno-suppressed individuals using an infusion of autologous peripheral blood lymphocyte, T-cells, or activated T-cells, which have been removed from the individual prior to a naturally occuring or induced state of immunosuppression. Such T-cells may have been activated by a CD3×CD28-based XCELLERATE process and/or may also have incorporated genes, proteins or be delivered concurrent with or sequentially with other molecules that have an additional benefit in modulating the immune system, tailoring T cell survival, tailoring T cell activity and thereby protecting an immuno-deficient individual.

Such methodologies may be extremely useful in patients who receive a myeloablative insult and whose immune system then requires recovery of the entire hematopoietic system, or portions thereof, from the bone marrow stem cell compartment. Alternatively, this would be useful in patients who are diagnosed with an immuno-deficiency and for whom a suitable donor source may or may not be found.

Patients who receive a myeloablative, lympho-ablative, or T cell-supressive insult experience a period often>1 year in which their immune system is deficient and recovery is dependent on restoration of either the entire hemtaopoietic system, or components of the immune system (e.g., T-cells) from differentiation of stem and progenitor cells in the bone marrow compartment. In a separate setting, some patients are diagnosed with naturally or environmentally induced immune-deficiencies which are correlated to loss or diminution of their endogenous immune cell functions.

This application would restore immune function into immuno-deficient individuals providing them with the ability to fight opportunistic infections and potentially also to combat residual cancer or other infectious agents.

In one embodiment, such therapy could be given in multiple cycles from a single collection of blood/apheresis product that is processed in such a manner as to provide multiple doses.

In further embodiments, a variety of gene modifications/insertions could be made. Cells could be loaded or delivered with vaccines, immuno-modulatory molecules, enzymes, chemical agents. Such “additives” could be administered along with the cells, prior to cell infusion, or after cell infusion, in order to enhance cell immune function, engraftment, survival and the like.

1. Ex Vivo T-Cell Expansion

The present invention provides methods for restoring or enhancing the immune system. In one aspect of the present invention, antibodies to CD3 and CD28 are co-immobilized on a solid surface. A preferred solid surface for such immobilization includes beads, and in certain aspects magnetic beads or beads capable of separation by filtration. In another aspect of the present invention, any ligand that binds the TCR/CD3 complex and initiates a primary activation signal may be utilized as a primary activation agent immobilized on the solid surface. In this regard, any ligand that binds CD28 and initiates the CD28 signal transduction pathway and which upon co-stimulation of the cell with a CD3 ligand induces a population of T-cells to proliferate is a CD28 ligand and accordingly, is a co-stimulatory agent within the context of the present invention.

a. The Primary Signal

The biochemical events responsible for ex vivo T-cell expansion are set forth briefly below. Interaction between the T-cell receptor (TCR)/CD3 complex and antigen presented in conjunction with either major histocompatibility complex (MHC) class I or class II molecules on an antigen-presenting cell initiates a series of biochemical events termed antigen-specific T-cell activation. Accordingly, activation of T-cells can be accomplished by stimulating the T-cell TCR/CD3 complex or via stimulation of the CD2 surface protein. An anti-CD3 monoclonal antibody can be used to activate a population of T-cells via the TCR/CD3 complex. A number of anti-human CD3 monoclonal antibodies are commercially available, exemplary are OKT3 prepared from hybridoma cells obtained from the American Type Culture Collection and monoclonal antibody G19-4. Similarly, stimulatory forms of anti-CD2 antibodies are known and available. Stimulation through CD2 with anti-CD2 antibodies is typically accomplished using a combination of at least two different anti-CD2 antibodies. Stimulatory combinations of anti-CD2 antibodies which have been described include the following: the T11.3 antibody in combination with the T11.1 or T11.2 antibody (Meuer et al., Cell 36:897-906, 1984) and the 9.6 antibody (which recognizes the same epitope as T11.1) in combination with the 9-1 antibody (Yang et al., J. Immunol. 137:1097-1100, 1986). Other antibodies which bind to the same epitopes as any of the above described antibodies can also be used. Additional antibodies, or combinations of antibodies, can be prepared and identified by standard techniques.

A primary activation signal can also be delivered to a T-cell via other mechanisms, for example through the use of a combination of a protein kinase C (PKC) activator such as a phorbol ester (e.g., phorbol myristate acetate) and a calcium ionophore (e.g., ionomycin which raises cytoplasmic calcium concentrations), or the like. The use of such agents bypasses the TCR/CD3 complex but delivers a stimulatory signal to T-cells.

b. The Secondary Signal

While, stimulation of the TCR/CD3 complex or CD2 molecule appears to be required for delivery of a primary activation signal in a T-cell, a number of molecules on the surface of T-cells, termed accessory or co-stimulatory molecules have been implicated in regulating the transition of a resting T-cell to blast transformation, and subsequent proliferation and differentiation. Thus, in addition to the primary activation signal, induction of T-cell responses requires a second, co-stimulatory signal. One such co-stimulatory or accessory molecule, CD28, is believed to initiate or regulate a signal transduction pathway that is distinct from those stimulated by the TCR complex.

Therefore, in order to induce an activated population of T-cells to proliferate in the absence of exogenous growth factors or accessory cells, an accessory molecule on the surface of the T-cell, such as CD28, is stimulated with a ligand that binds the accessory molecule. In one embodiment, stimulation of the accessory molecule CD28 and activation occurs simultaneously by contacting a population of T-cells with a ligand that binds CD3 and a ligand that binds CD28. Activation of the T-cells with, for example, an anti-CD3 antibody and stimulation of the CD28 accessory molecule results in selective proliferation of CD4⁺ T-cells.

Accordingly, one of ordinary skill in the art will recognize that any agent, including an anti-CD28 antibody or fragment thereof capable of crosslinking the CD28 molecule, or a natural ligand for CD28 can be used to stimulate T-cells. Exemplary anti-CD28 antibodies or fragments thereof useful in the context of the present invention include monoclonal antibody 9.3 (Bristol-Myers Squibb, Stamford, Conn.)—an IgG2a antibody, monoclonal antibody KOLT-2—an IgG1 antibody, 15E8—an IgG1 antibody, 248.23.2—an IgM antibody and EX5.3D10 (ATCC#HB11373)—an IgG2a antibody. Exemplary natural ligands include the B7 family of proteins, such as B7-1 (CD80) and B7-2 (CD86) (Freedman et al., J. Immunol. 137:3260-3267, 1987; Freeman et al., J. Immunol. 143:2714-2722, 1989; Freeman et al., J. Exp. Med. 174:625-631, 1991; Freeman et al., Science 262:909-911, 1993; Azuma et al., Nature 366:76-79, 1993; Freeman et al., J. Exp. Med. 178:2185-2192, 1993). In addition, binding homologues of a natural ligand, whether native or synthesized by chemical or recombinant techniques, can also be used in accordance with the present invention.

c. Primary and Secondary Stimulation

Further, it should be understood that ligands useful for stimulating an accessory molecule can be used in soluble form, attached to the surface of a cell, or immobilized on a solid surface as described herein, it is preferred that both primary and secondary signals are co-immobilized on a solid surface. In one aspect, the molecule providing the primary activation signal (e.g., TCR/CD3 ligand) and the co-stimulatory molecule (e.g., CD28 ligand) are coupled to the same solid phase support (e.g., a bead).

In one aspect of the present invention, ex vivo T-cell expansion can be performed by isolation of T-cells and subsequent stimulation and expansion utilizing a “surrogate” APC (e.g., a ligand conjugated solid surface). A source of T-cells is obtained from a subject. The term “subject” is intended to include living organisms in which an immune response can be elicited (e.g., mammals). Examples of subjects include humans, dogs, cats, mice, rats, and transgenic species thereof. T-cells can be obtained from a number of sources, including peripheral blood leukocytes, bone marrow, lymph node tissue, spleen tissue, and tumors. Preferably, peripheral blood leukocytes are obtained from an individual by leukopheresis (e.g., apheresis). To isolate T-cells, if desired, from peripheral blood leukocytes, it may be necessary to lyse the red blood cells and separate peripheral blood leukocytes from monocytes by, for example, centrifugation through a PERCOLL™ gradient. A specific subpopulation of T-cells, such as CD28⁺, CD4⁺, CD8⁺, CD28RA⁺, and CD28RO⁺T-cells, can be further isolated by positive or negative selection techniques. For example, negative selection of a T-cell population can be accomplished with a combination of antibodies directed to surface markers unique to the cells negatively selected. A preferred method is cell sorting via negative magnetic immunoadherence which utilizes a cocktail of monoclonal antibodies directed to cell surface markers present on the cells negatively selected. For example, to isolate CD4⁺ cells, a monoclonal antibody cocktail typically includes antibodies to CD14, CD20, CD11b, CD16, HLA-DR, and CD8.

The process of negative selection results in an essentially homogenous population of CD28⁺, CD4⁺ or CD8⁺ T-cells. The T-cells can be activated as described herein, such as by contact with a anti-CD3 antibody immobilized on a solid phase surface or an anti-CD2 antibody, or by contact with a protein kinase C activator (e.g., bryostatin) in conjunction with a calcium ionophore. To stimulate an accessory molecule on the surface of the T-cells, a ligand which binds the accessory molecule is employed. For example, a population of CD4⁺ cells can be contacted with an anti-CD3 antibody and an anti-CD28 antibody, under conditions appropriate for stimulating proliferation of the T-cells. Similarly, to stimulate proliferation of CD8⁺ T-cells, an anti-CD3 antibody and the monoclonal antibody ES5.2D8 (ATCC) can be used as can other methods commonly known in the art (Berg et al., Transplant Proc. 30(8):3975-3977, 1998; Haanen et al., J. Exp. Med. 190(9):1319-1328, 1999; Garland et al., J. Immunol Meth. 227(1-2):53-63, 1999). Conditions appropriate for T-cell culture include an appropriate media (e.g., Minimal Essential Media or RPMI Media 1640) which may contain factors necessary for proliferation and viability, including animal serum (e.g., fetal bovine serum) and antibiotics (e.g., penicillin streptomycin). The T-cells are maintained under conditions necessary to support growth, for example an appropriate temperature (e.g., 37° C.) and atmosphere (e.g., air plus 5% CO₂).

The primary activation signal and the co-stimulatory signal for the T-cell can be provided by different protocols. For example, the agents providing each signal can be in solution or coupled to a solid surface. When coupled to a solid surface, the agents can be coupled to the same solid surface (i.e., in “cis” formation) or to separate surfaces (i.e., in “trans” formation). Alternatively, one agent can be coupled to a solid surface and the other agent in solution. In one embodiment, the agent providing the co-stimulatory signal is bound to a cell surface and the agent providing the primary activation signal is in solution or coupled to a solid phase surface. In a preferred embodiment, the two agents are immobilized on beads, either on the same bead, i.e., in “cis,” or to separate beads, i.e., in “trans.” Alternatively, the agent providing the primary activation signal is an anti-CD3 antibody and the agent providing the co-stimulatory signal is an anti-CD28 antibody; both agents are co-immobilized to the same bead. In this embodiment, a preferred ratio of each antibody bound to the beads for CD4⁺ T-cell expansion and T-cell growth is a 1:1 ratio. However, ratios from 1:9 to 9:1 can also be used to stimulate T-cell expansion. The ratio of anti-CD3 and anti-CD28 coupled (with a ratio of 1:1 of each antibody) beads to T-cells that yield T-cell expansion can vary from 1:3 to 3:1, with the optimal ratio being 3:1 beads per T-cell. Moreover, it has been determined that when T-cells are expanded under these conditions, they remain polyclonal.

To maintain long term stimulation of a population of T-cells following the initial activation and stimulation, it may be necessary to separate the T-cells from the stimulus after a period of about 12 to about 14 days. The rate of T-cell proliferation is monitored periodically (e.g., daily) by, for example, examining the size or measuring the volume of the T-cells, such as with a Coulter Counter. In this regard, a resting T-cell has a mean diameter of about 6.8 microns, and upon initial activation and stimulation, in the presence of the stimulating ligand, the T-cell mean diameter will increase to over 12 microns by day 4 and begin to decrease by about day 6. When the mean T-cell diameter decreases to approximately 8 microns, the T-cells may be reactivated and re-stimulated to induce further proliferation of the T-cells. Alternatively, the rate of T-cell proliferation and time for T-cell re-stimulation can be monitored by assaying for the presence of cell surface molecules, such as B7-1, B7-2, which are induced on activated T-cells.

For inducing long term stimulation of a population of CD4⁺ and/or CD8⁺ T-cells, it may be necessary to reactivate and re-stimulate the T-cells with a anti-CD3 antibody and an anti-CD28 antibody or monoclonal antibody ES5.2D8 several times to produce a population of CD4⁺ or CD8⁺ cells increased in number from about 10 to about 1,000-fold the original T-cell population. Using this methodology, it is possible to get increases in a T-cell population of from about 100 to about 100,000-fold an original resting T-cell population.

In the various embodiments, one of ordinary skill in the art understands that removal of the stimulation signal from the cells is dependent upon the type of solid surface used. For example, if paramagnetic beads are utilized then magnetic separation is a feasible option. Such separation techniques are described in detail by paramagnetic bead manufactures instructions (see, e.g., DYNAL Inc.) Further, if the solid surface is a bead large enough to be separated from the cells by filtration, this method may also be employed. Briefly, a variety of transfusion filters are commercially available, including 20 micron and 80 micron transfusion filters sold by Baxter. Accordingly, as long as the beads are larger than the mesh size of the filter, such filtration is highly efficacious. Further, given that these simple filtration systems are clinical approved the impact of further FDA scrutiny of the ex vivo methodology should be minimal.

Although the antibodies used in the methods described herein can be readily obtained from public sources, such as the ATCC, antibodies to T-cell surface accessory molecules and the CD3 complex can be produced by standard techniques. Methodologies for generating antibodies for use in the methods of the invention are well known in the art and are discussed in further detail below.

2. Dual Ligand Immobilization On a Solid Surface

As indicated above, certain methods of the present invention preferably utilize ligands bound to a solid surface. The solid surface to be used can be any solid surface that is capable of having a ligand bound thereto and which is also non-toxic to the T-cells to be stimulated. For example, the solid surface may comprise cellulose, agarose, polyacrylamide, acrolein, dextran, any number of plastics or the like. In the various embodiments, commercially available solid surfaces such as beads are preferred (e.g., Sepharose beads, Pharmnacia Fine Chemicals, Sweden; DYNABEADS™, Dynal Inc., New York; Purabeads™, Prometic Biosciences™).

When beads are utilized, the bead can be of any size that effectuates T-cell expansion. In one embodiment beads are preferably from 2.8 μm to 500 μm in size. Accordingly, the choice of bead size depends on the particular use the bead will serve. Further, when paramagnetic beads are employed the beads typically range in size from about 2.8 μm to about 500 μm and more preferably from about 2.8 μm to about 50 μm.

In addition to composition and size of the solid surface, an additional consideration is the coupling of the ligand thereto. Ligands may be coupled to the solid surface by a variety of methods known and available in the art. The terms “coupled” or “coupling” refer to a chemical, enzymatic or other means (e.g., antibody, avidin/streptavidin-biotin) by which the ligand (e.g., anti-CD3 and anti-CD28) is linked to a solid surface such that the ligand is present on the solid surface and is capable of triggering activation and proliferation. For example, protein A coated solid surfaces may be utilized to bind an antibody, or the ligand may be immobilized on the surface by chemical means such as crosslinking to the solid surface using commercially available crosslinking reagents (Pierce, Rockford IL) or other means. In preferred embodiments, the ligands are covalently bound to the solid surface. Further, in one embodiment, commercially available tosylactivated DYNABEADS™ or DYNABEADS™ with epoxy-surface reactive groups are incubated with the polypeptide ligand of interest according to the manufacturer's instructions. Briefly, such conditions are typically incubation in a phosphate buffer from pH 4 to pH 9.5 and with temperatures ranging from 4 to 37 degrees C.

In one aspect both ligands are antibodies or fragments thereof while in another aspect, the co-stimulatory ligand is a B7 molecule (e.g., B7-1, B7-2) which are coupled to the solid surface by any of a variety of the different methods discussed above. In this aspect, the B7 molecule to be coupled to the solid surface can be obtained using standard recombinant DNA technology and expression systems that allow for production and isolation of the co-stimulatory molecule(s) or obtained from a cell expressing the co-stimulatory molecule, as described herein. Fragments, mutants or variants of a B7 molecule which retain the ability to trigger a co-stimulatory signal in T-cells when coupled to the surface of a cell can also be used. Furthermore, one of ordinary skill in the art will recognize that the ligands used to activate and induce proliferation of a subset of T-cells may also be immobilized on beads or culture vessel surfaces. In addition, while covalent binding of the ligand to the solid surface is preferred, adsorption or capture by a secondary monoclonal antibody may also be utilized.

The amount of a particular ligand attached to the solid phase surface can be readily determined by FACS analysis if the solid surface is that of beads or by ELISA if the solid phase surface is that of a tissue culture dish.

In a particular embodiment, the stimulatory form of a B7 molecule or an anti-CD28 antibody or fragment thereof is attached to the same solid phase surface as the agent that stimulates the TCR/CD3 complex, such as an anti-CD3 antibody. In addition to anti-CD3, other antibodies that bind to receptors that mimic antigen signals may be used, for example, the beads or other solid phase surface may be coated with combinations of anti-CD2 and a B7 molecule and in particular anti-CD3 and anti-CD28.

3. Ligands

In one aspect of the present invention, useful ligands include agents that are capable of binding the CD3/TCR complex or CD28 and initiating activation or proliferation, respectively. Accordingly, the term ligand includes those proteins which are the “natural” ligand for the cell surface protein, such as a B7 molecule for CD28, as well as artificial ligands such as antibodies directed to the cell surface protein. Such antibodies and fragments thereof may be produced in accordance with conventional techniques, such as hybridoma synthesis, recombinant DNA techniques and protein synthesis. Useful antibodies and fragments may be derived from any species (including humans) or may be formed as chimeric proteins which employ sequences from more than one species. See, generally, Kohler and Milstein, Nature 256: 495-597, 1975; Eur. J. Immunol. 6: 511-519, 1976.

Antibodies to CD3 and CD28, fragments, or peptides discussed herein may readily be prepared. Within the context of the present invention, antibodies are understood to include monoclonal antibodies, polyclonal antibodies, anti-idiotypic antibodies, antibody fragments (e.g., Fab, and F(ab′)₂, Fᵥ variable regions, or complementarity determining regions). Antibodies are generally accepted as specific against the particular antigen if they bind with a K_{d} of greater than or equal to 10⁻⁷ M, preferably greater than of equal to 10⁻⁸ M. The affinity of a monoclonal antibody or binding partner can be readily determined by one of ordinary skill in the art (see Scatchard, Ann. N.Y. Acad. Sci. 51:660-672, 1949).

Briefly, a polyclonal antibody preparation may be readily generated in a variety of warm-blooded animals such as rabbits, mice, or rats. Typically, an animal is immunized with the appropriate protein (e.g., CD3 or CD28) or peptide thereof, which may be conjugated to a carrier protein, such as keyhole limpet hemocyanin. Routes of administration include intraperitoneal, intramuscular, intraocular, or subcutaneous injections, usually in an adjuvant (e.g., Freund's complete or incomplete adjuvant). Particularly preferred polyclonal antisera demonstrate binding in an assay that is at least three times greater than background.

Monoclonal antibodies may also be readily generated from hybridoma cell lines using conventional techniques (see U.S. Pat. Nos. RE 32,011, 4,902,614, 4,543,439, and 4,411,993; see also Antibodies: A Laboratory Manual, Harlow and Lane (eds.), Cold Spring Harbor Laboratory Press, 1988). Briefly, within one embodiment, a subject animal such as a rat or mouse is injected with the appropriate protein (e.g., CD3 or CD28) or a portion thereof. The protein may be administered as an emulsion in an adjuvant such as Freund's complete or incomplete adjuvant in order to increase the immune response. Between one and three weeks after the initial immunization the animal is generally boosted and may tested for reactivity to the protein utilizing well-known assays. The spleen and/or lymph nodes are harvested and immortalized. Various immortalization techniques, such as mediated by Epstein-Barr virus or fusion to produce a hybridoma, may be used. In a preferred embodiment, immortalization occurs by fusion with a suitable myeloma cell line (e.g., NS-1 (ATCC No. TIB 18), and P3X63-Ag 8.653 (ATCC No. CRL 1580) to create a hybridoma that secretes monoclonal antibody. The preferred fusion partners do not express endogenous antibody genes. Following fusion, the cells are cultured in medium containing a reagent that selectively allows for the growth of fused spleen and myeloma cells such as HAT (hypoxanthine, aminopterin, and thymidine) and are subsequently screened for the presence of antibodies that are reactive against the protein of interest (e.g., CD3 or CD28). A wide variety of assays may be utilized, including for example countercurrent immuno-electrophoresis, radioimmunoassays, radioimmunoprecipitations, enzyme-linked immunosorbent assays (ELISA), dot blot assays, western blots, immunoprecipitation, inhibition or competition assays, and sandwich assays (see U.S. Pat. Nos. 4,376,110 and 4,486,530; see also Antibodies: A Laboratory Manual, Harlow and Lane (eds.), Cold Spring Harbor Laboratory Press, 1988).

Other techniques may also be utilized to construct monoclonal antibodies (see Huse et al., Science 246:1275-1281, 1989; Sastry et al., Proc. Natl. Acad. Sci. USA 86:5728-5732, 1989; Alting-Mees et al., Strategies in Molecular Biology 3:1-9, 1990; describing recombinant techniques). Briefly, RNA is isolated from a B cell population and utilized to create heavy and light chain immunoglobulin cDNA expression libraries in suitable vectors, such as λImmunoZap(H) and λImmunoZap(L). These vectors may be screened individually or co-expressed to form Fab fragments or antibodies (see Huse et al., supra; Sastry et al., supra). Positive plaques may subsequently be converted to a non-lytic plasmid that allows high level expression of monoclonal antibody fragments from E. coli.

Similarly, portions or fragments, such as Fab and Fv fragments, of antibodies may also be constructed utilizing conventional enzymatic digestion or recombinant DNA techniques to yield isolated variable regions of an antibody. Within one embodiment, the genes which encode the variable region from a hybridoma producing a monoclonal antibody of interest are amplified using nucleotide primers for the variable region. These primers may be synthesized by one of ordinary skill in the art, or may be purchased from commercially available sources (e.g. Stratacyte, La Jolla, Calif.) Amplification products are inserted into vectors such as ImmunoZAP™ H or ImmunoZAP™ L (Stratacyte), which are then introduced into E. coli, yeast, or mammalian-based systems for expression. Utilizing these techniques, large amounts of a single-chain protein containing a fusion of the V_{H} and V_{L} domains may be produced (see Bird et al., Science 242:423-426, 1988). In addition, techniques may be utilized to change a “murine” antibody to a “human” antibody, without altering the binding specificity of the antibody.

Human monoclonal antibodies or “humanized” murine antibody are also useful as ligands in accordance with the present invention. For example, murine monoclonal antibody may be “humanized” by genetically recombining the nucleotide sequence encoding the murine Fv region (i.e., containing the antigen binding sites) or the complementarily determining regions thereof with the nucleotide sequence encoding a human constant domain region and an Fc region, e.g., in a manner similar to that disclosed in European Patent Application No. 0,411,893 A2.

Humanized antibodies may be produced by a variety of methods. These humanization methods include: (a) grafting only non-human CDRs onto human framework and constant regions (e.g., Jones et al., Nature 321:522-525 (1986) (conventional humanized antibodies); Verhoeyen et al., Science 239:1534-1536 (1988); and (b) transplanting the entire non-human variable domains, but cloaking (veneering) these domains by replacement of exposed residues (to reduce immunogenicity) (e.g., Padlan, Molec. Immun. 28:489-498 (1991) (veneered antibodies).

In particular, the present invention embraces substitution modifications which do not substantially adversely affect antigen binding. For example, this includes conservative amino acid substitutions, e.g, the substitution of an acidic amino acid by another acidic amino acid. Conservative amino acid substitution mutations are well known in the art.

In addition, those of ordinary skill in the art will recognize that an antibody within the context of the present invention may be truncated by the deletion of one or more amino acid residues to produce functional (antigen-binding) sequences. Functional deletions can be identified by sequential expression of various deletions, and screening the resultant deletion to determine its ability to bind the requisite antigen (e.g., CD3 or CD28). As described below, mutated antibody sequences may be expressed in any of a variety of host systems, e.g., mammalian cells (such as CHO cells), insects, planT-cells, transgenic plants and transgenic animals. In this regard it should be understood that antibody or antibody fragment sequences may be derived from a certain source, be it murine, human, or the like, yet the original sequence has been modified by substitution, deletion, or addition, yet retains the same or greater binding affinity for the original epitope as compared to the protein encoded by the original sequence.

The antibodies useful within the context of the present invention may be obtained by expression of whole immunoglobulin or fragments thereof including variable heavy and light chains, such as scFv, in an appropriate host system. Essentially, as used herein an appropriate “host system” refers to any expression system including host cell tissue or multicellular organism and vector or vectors containing nucleic acid sequences which encode the subject antibodies or fragments thereof, which in combination provide for the expression of functional antibodies, i.e., the heavy and light chains associate to produce the characteristic antigen-binding structure.

The following references are representative of methods and host systems suitable for expression of recombinant immunoglobulins: Weidle et al., Gene 51:21-29, 1987; Dorai et al., J. Immunol. 13(12):4232-4241, 1987; De Waele et al., Eur. J. Biochem. 176:287-295, 1988; Colcher et al., Cancer Res. 49:1738-1745, 1989; Wood et al., J. Immunol. 145(a):3011-3016, 1990; Bulens et al., Eur. J. Biochem. 195:235-242, 1991; Beggington et al., Biol. Technology 10:169, 1992; King et al., Biochem. J. 281:317-323, 1992; Page et al., Biol. Technology 9:64, 1991; King et aL, Biochem. J. 290:723-729, 1993; Chaudary et al., Nature 339:394-397, 1989; Jones et al., Nature 321:522-525, 1986; Morrison and Oi, Adv. Immunol. 44:65-92, 1988; Benhar et al., Proc. Natl. Acad. Sci. USA 91:12051-12055, 1994; Singer et al., J. Immunol. 150:2844-2857, 1993; Cooto et al., Hybridoma 13(3):215-219, 1994; Queen et al., Proc. Natl. Acad. Sci. USA 86:10029-10033, 1989; Caron et aL, Cancer Res. 32:6761-6767, 1992; Cotoma et al., J. Immunol. Meth. 152:89-109, 1992.

Expression host systems including vectors, hosT-cells, tissues and organisms capable of producing functional recombinant antibodies, and in particular humanized and chimeric antibodies, are well known in the art. Moreover, host systems suitable for expression of recombinant antibodies are commercially available.

Host cells known to be capable of expressing immunoglobulins or antibody fragments include, by way of example, mammalian cells such as Chinese Hamster Ovary (CHO) cells, COS cells, myeloma cells; bacteria such as Escherichia coli; yeast cells such as Saccharomyces cerevisiae; insect-cells such as Spodoptera frugiperda; among other host-cells. CHO cells are used by many researchers given their ability to effectively express and secrete immunoglobulins. Also, insect cells are desirable because they are capable of high expression of recombinant proteins.

In addition, antibodies may be expressed in transgenic plants (see, e.g., U.S. Pat. No. 5,202,422) or animals. The subject antibody sequences may be operatively linked to a promoter that is specifically activated in mammary tissue such as a milk-specific promoter. Such methods are described in U.S. Pat. Nos. 4,873,316 and 5,304,498, incorporated herein. Typically, such methods will use a vector containing a signal peptide which enables secretion of an operably linked polypeptide sequence, a milk specific promoter such as casein promoter, an enhancer sequence and immunoglobulin sequences specific to the requisite ligand.

This vector will be introduced in a suitable host, e.g., bovine, ovine, porcine, rabbit, rat, frog, or mouse embryo, typically by microinjection under conditions whereby the expression vector integrates into the genome of the particular embryo. The resultant transgenic embryo is then transferred to a surrogate mother, and offspring are screened to identify those transgenics which contain and express the antibodies in their milk. Transgenics which contain and/or express the antibody sequences may be identified, e.g., by Southern blot or Western blot analysis. The milk produced by such transgenic animals is then collected and the antibodies isolated therefrom. As noted, such methods are described in detail in U.S. Pat. Nos. 4,873,316 and 5,304,498.

Recombinant expression of functional antibody fragments may be effected by one of three general methods. In the first method, the host or host cells are transfected with a single vector which provides for the expression of both heavy and light variable sequences fused to appropriate constant regions. In the second method, host cells are transfected with two vectors, which respectively provide for expression of either the variable heavy or light sequence fused to an appropriate constant region. In the third method the host or host cells are transfected with a single vector which provides for the expression of both heavy and light variable sequences fused by an appropriate linker, thereby encoding a single chain antibody or scFv.

In expressing recombinant antibodies in cell culture, e.g., in CHO cells or insect cells, the expression system (e.g., expression vector) will preferably contain sequences which provide for the selection of transfectants and expression of the antibodies. Therefore, preferably the vector or vectors will contain genes which allow for selection, e.g., antibiotic (or drug) resistance genes. Also, the vector will preferably contain promoters which provide for efficient expression of the heavy and light chains as well as other regulatory sequences, e.g., polyadenylation regions, enhancer regions, etc. The design of systems suitable for expression of recombinant antibodies is well known and within the purview of one of ordinary skill in the art, as evidenced by the above-identified references relating to expression of recombinant immunoglobulins.

A well known example of host cells suitable for expression of immunoglobulins is CHO cells. In expressing immunoglobulins in CHO cells, or other mammalian cells, it is desirable to include a sequence which provides for amplification, so as to enhance vector copy number and enhance antibody yields. Such sequences, includes, by way of example dominant selectable markers, such as dihydrofolate reductase (DHFR), neomycin phosphotransferase (Neo), glutamine synthetase (GS), adenosine deaminase (ADA), among others.

Examples of suitable promoters useful for the expression of proteins in mammalian cells include, by way of example, viral promoters such as the human cytomegalovous (CMV) early promoter, SV40 early and late promoters, and the RSV promoter and enhancer. Also, mammalian promoters may be used, e.g., immunoglobulin promoters, growth hormone promoters such as bovine growth hormone promoter, etc. It is preferable to select a strong promoter, i.e., one which provides for high levels of transcription.

Also, the vector will preferably contain polyadenylation sequences (polyA) sequences which provide for polyadenylation of mRNA which function to enhance mRNA stability, and thereby enhance protein production. Examples of suitable poly A sequences include, by way of example, SV40 poly A sequences, and bovine growth hormone promoter (BGH) poly A sequence, among others.

After the subject antibodies are expressed they are purified and then assayed for their ability to bind antigen. Methods for purifying recombinant immunoglobulins are well known and are described in the references incorporated herein relating to production of recombinant antibodies. For example, a well known method of purifying antibodies involves protein A purification because of the propensity of protein A to bind the Fc region of antibodies. In addition, columns containing the antigen of interest may be used to purify the desired antibody.

One of ordinary skill in the art will appreciate that a variety of alternative techniques for generating antibodies exist. In this regard, the following U.S. patents teach a variety of these methodologies and are thus incorporated herein by reference: U.S. Pat. Nos. 5,840,479; 5,770,380; 5,204,244; 5,482,856; 5,849,288; 5,780,225; 5,395,750; 5,225,539; 5,110,833; 5,693,762; 5,693,761; 5,693,762; 5,698,435; and 5,328,834.

Once suitable antibodies have been obtained, they may be isolated or purified by many techniques well known to those of ordinary skill in the art (see Antibodies: A Laboratory Manual, Harlow and Lane (eds.), Cold Spring Harbor Laboratory Press, 1988). Suitable techniques include peptide or protein affinity columns, HPLC (e.g., reversed phase, size exclusion, ion-exchange), purification on protein A or protein G columns, or any combination of these techniques.

Thus, it is routine to test the ability of antibodies produced according to recombinant or hybridoma methodologies for binding to the T-cell anti-ligand of interest (e.g., CD3 and CD28), as methods for evaluating the ability of antibodies to bind to epitopes of these antigens are known.

B. Pharmaceutical Compositions

Lymphocytes, T-cells, activated T-cells, activated and gene modified T-cells, and/or activated and otherwise modulated T-cells of the present invention may be administered either alone, or as a pharmaceutical composition. Briefly, pharmaceutical compositions of the present invention may comprise a cell population as described herein, in combination with one or more pharmaceutically or physiologically acceptable carriers, diluents or excipients. Such compositions may comprise buffers such as neutral buffered saline, phosphate buffered saline and the like, carbohydrates such as glucose, mannose, sucrose or dextrans, mannitol, proteins, polypeptides or amino acids such as glycine, antioxidants, chelating agents such as EDTA or glutathione, adjuvants (e.g., aluminum hydroxide) and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

All references referred to within the text are hereby incorporated by reference in their entirety. Further, the following examples are offered by way of illustration, and not by way of limitation.

### EXAMPLES

### Example 1

### Lympgocyte or T-Cell Collection and Culture

Cells isolated from human blood are grown in X-vivo media (Biowhittaker Inc., Walkersville, Md.) and depending on use supplemented with or without 20 U/ml IL-2 (Boehringer Mannheim, Indianapolis, IN) and supplemented with 5% human serum (Biowhittaker), 2 mM Glutamine (Life Technologies, Rockville, Md.) and 20 mM HEPES (Life Technology). Jurkat E6-1 cells (ATCC, Manassas, Va.) are grown in RPMI 1640 (Life Technologies) supplemented with 10% FBS (Biowhittaker), 2 mM glutamine (Life Technologies), 2 mM Penicillin (Life Technologies), and 2 mM Streptomycin (Life Technologies).

Buffy coats from healthy human volunteer donors are obtained (American Red Cross, Portland, Oreg.). Peripheral blood mononuclear cells (PBMC) are obtained using Lymphocyte Separation Media (ICN Pharmaceuticals, Costa Mesa, Calif.) according to the manufacturers' instructions.

Peripheral blood lymphocytes (PBL) are obtained from the PBMC fraction by incubation in culture flask (Costar, Pittsburgh, Pa.) with uncoated DYNABEADS™ (Dynal, Oslo, Norway), 10⁸ cells/ml, 2 beads/cell, 2 h at 37° C. Monocytes and macrophages stick to the culture flask or phagocytose the paramagnetic beads that are depleted by magnetic cell separation according to the manufacture's instruction (Dynal). CD4 cells are purified from the PBL fraction by incubation with 10 μg/ml of monoclonal antibodies against CD8 (clone G10-1), CD20 (clone IF5), CD14 (clone F13) and CD16 (Coulter), 10⁸ cells/ml, 20 min at 4° C. After washing, cells are depleted twice with sheep anti-mouse Ig-coupled DYNABEADS™ (10⁶ cells/ml, 6 beads/cell, 20 min at 4° C.) and magnetic cell separation. The purity of CD4 cells are routinely 91-95% as measured by Flow cytometry.

Dendritic cells are generated from PBMC adhering to the culture flask (Costar), 10⁸ cells/ml, 2 h at 37° C. (without DYNABEADS™). After extensive washing, adherent cells are cultured for 7 days in media containing 500 U/ml GM-CSF (Boehringer Mannheim) and 12.5 U/ml EL-4 (Boehringer Mannheim). The resulting cell population is weakly adherent and expresses surface markers characteristic of dendritic cells (Positive for HLA-DR, CD86, CD83, CD11c and negative for CD4). (All antibodies obtained from Becton Dickinson, Calif.).

The anti-CD3 mAb (OKT3) is obtained from Ortho Biotech., (Raritan, N.J.) and the anti-CD28 mAb (9.3) is obtained from Bristol-Myers Squibb, (Stamford, Conn.).

Other techniques can utilize human peripheral blood lymphocytes containing T-cells are incubated in tissue culture plates and/or tissue culture flasks (Baxter bags), or other common culture vessels in media which could be composed of RPMI, X-Vivo 15, or some other T-cell culture media. Although not required for the activation and growth of T-cells, glutamine and HEPES are added to the culture media. Fetal bovine serum (10% final), human A/B serum (5%), or autologous human serum (5%) is added to culture media. The percentage of serum may vary without greatly affecting T-cell biology or culture outcome. In some instances, recombinant human IL2 is added to cultures. In some instances, phagocytic CD14+ cells and other phagocytic cells are remove by magnetic depletion as described, infra. Beads having co-immobilized upon their surface anti-CD3 and anti-CD28 (3×28 beads) are added at a 3:1 bead:cell ratio. Cultures are maintained at 37 degrees C. at 5-7% CO₂. Cells are removed at several timepoints over a 14 day period to determine cell density (cell number), cell size, and cell surface phenotype as measured via flow cytometric analysis of a variety of surface antigens. Supernatants are also collected from cultures to determine cytokine secretion profiles, including, but not limited to: IL2, IL4, IFN_{λ}, TNFα. As activated cells grow and divide, cultures are maintained at 0.2-2×10⁶ CD3+ T-cells/ml. When T-cell density exceeds roughly 1.5×10⁶/ml, cultures are split and fed with fresh media so as to give a cell density in the 0.2-1.4×10⁶/ml range. At roughly 2 hours to about 14 days following initial stimulation, when activated T-cells are shown to be entering a more quiescent phase (e.g., CD25 levels diminishing, cell size as determined by forward scatter is diminishing, rate of cell division may be reduced), cells are either reinfused into the subject or re-stimulated with one of the following stimuli:

1) No stimulus

2) Phytohemagglutinin (PHA) 2 μg/ml

3) (3×28 beads) at a 1:1 bead/cell ratio

Cells are again analyzed over time for cell phenotype and activation/functional state. Supernatants are again collected for secreted cytokine analysis.

### Example 2

### Activation and Cell Proliferation Assays

Cells are stimulated by three different methodologies 1) Dynal beads (M-450) covalently coupled to anti-CD3 (OKT-3) and anti-CD28 (9.3) antibodies (3×28 beads) according to the manufacturer's instructions (Dynal), 3 beads/cell, 2) lonomycin (Calbiochem, La Jolla, Calif.) (100 ng/ml) and phorbol 12-myristate-13-acetate (PMA) (Calbiochem) (10 ng/ml), 3) allogeneic dendritic cells (25,000 dendritic cells/200,000 CD4 cells). All cells are stimulated at a concentration of 10⁶ cell/ml. Proliferation assays are conducted in quadruplicate in 96 well flat-bottom plates. Cells are stimulated at 10⁶ cells/ml in a final volume of 200 μl. Proliferation is measured by MTT assay (MTT assay kit, Chemicon International Inc., Temecula, Calif.) at day 3 (stimulation method 1 and 2) or at day 6 (stimulation method 3), and results are presented as mean value of quadruplicates. PBL cultures or purified CD4 cell cultures are stimulated with 3×28 beads, ionomycin/PMA or allogenic dendritic cells.

### Example 3

### Radioisotope T-Cell Proliferation Assays

Peripheral blood mononuclear cells (PBMC) from healthy donors are separated by density centrifugation with ficoll-hypaque (LSM, Organon Teknika, Durham, N.C.). After washing the PBMC with complete media (RPMI 1640 medium with 5% human serum, 100 mM glutamine, 1 mM sodium pyruvate, 0.1 mM non-essential amino acid, 2 mM Penicillin (Life Technologies), and 2 mM Streptomycin (Life Technologies), they are then irradiated at 7,500 RADS, and resuspended at 4-4.5×10⁶ cells/ml in complete media. Another aliquot of PBMC are rosetted with neuraminidase treated SRBC. After another centrifugation with LSM, the sheep red blood cells (SRBC) of these rosetted T-cells are then lysed with ammonium chloride lysing buffer (Life Technologies). After washing 2× with complete media, these purified T-cells are also resuspended at 2-2.5×10⁶ cells/ml in complete media. The various dilutions of the compound are added in triplicates at 50 μl/well of a 96 well flat-bottom microculture plate (Costar, Cambridge, Mass.). The T-cell suspension is then immediately distributed into the wells at 100 μl/well. After incubating the cells with compound for 30 min. at 37° C. in a humidified atmosphere of 5% CO₂-95% air, 20 μl/well of anti-CD3 (OKT-3, Ortho Diagnostic, N.J.) at final conc. of 10 ng/ml is added, followed by 50 μl of the irradiated PBMC. The culture plates are then incubated at 37° C. in a humidified atmosphere of 5% CO₂-95% air for 72 hours. The proliferation of T lymphocytes is assessed by measurement of tritiated thymidine incorporation. During the last 18-24 hours of culturing, the cells are pulse-labeled with 2 μCi/well of tritiated thymidine (NEN, Cambridge, Massachusetts). The cultures are harvested on glass fiber filters using a multiple sample harvester (MACH-II, Wallace, Gaithersburg, Md.). Radioactivity of filter discs corresponding to individual wells is measured by standard liquid scintillation counting methods (Betaplate Scint Counter, Wallace). Mean counts per minute of replicate wells are calculated and the results were expressed as concentration of compound required to inhibit tritiated thymidine uptake of T-cells by 50%.

From the foregoing it will be appreciated that, although specific embodiments of the invention have been described herein for purposes of illustration, various modifications may be made without deviating from the spirit and scope of the invention. Accordingly, the invention is not limited except as by the appended claims. All of references, patents, patent applications, etc. cited above, are incorporated herein in their entirety.

1. A method for restoration or enhancement of immune function in an immuno-compromised or immuno-suppressed animal, comprising administering cell population selected from the group consisting of peripheral blood lymphocytes, T-cells, or activated T-cells, thereby restoring or enhancing the immune function of the animal.

2. The method of claim 1 wherein said cell population is autologous.

3. The method of claim 2 wherein said cell population is activated by stimulation of the CD3 and CD28 cell surface markers.

4. The method of claim 3 wherein said stimulation is provided by anti-CD3 and anti-CD28 antibodies or fragments thereof.

5. The method of claim 1 wherein said cell population is allogeneic.
